(19) United States
(12) Patent Application Publication
(10) Pub. No.: US 2014/0285655 A1
(43) Pub. Date: Sep. 25, 2014

(54) APPARATUS AND METHOD FOR MEASURING SHAPE OF UNDERWATER OBJECT

(71) Applicant: Electronics and Telecommunications Research Institute, Daejeon (KR)

(72) Inventors: Seong-Ho SON, Daejeon (KR); Hyuk Je KIM, Daejeon (KR); Jong Moon LEE, Daejeon (KR); Bo Ra KIM, Daejeon (KR); Soon Ik JEON, Daejeon (KR); Hyung Do CHOI, Daejeon (KR)

(21) Appl. No.: 13/905,883

(22) Filed: May 30, 2013

(30) Foreign Application Priority Data
Mar. 20, 2013 (KR) 10-2013-0029659

(51) Int. Cl. G01B 11/24; A61B 5/00

(52) U.S. Cl. CPC G01B 11/24; A61B 5/0091
USPC 348/81; 600/407

(57) ABSTRACT

An apparatus for measuring a shape of an underwater object includes a laser emitter configured to irradiate a laser on a surface of a measurement target object under water; and a camera configured to capture a laser point generated at the surface of the measurement target object by the laser emitter. Further, the apparatus includes a shape restorer configured to convert a pixel coordinates value of the laser point captured by the camera into an absolute coordinates value to restore a three-dimensional (3D) shape of the measurement target object.

### CROSS-REFERENCE TO RELATED APPLICATION(S)

The present invention claims priority of Korean Patent Application No. 10-2013-0029659, filed on Mar. 20, 2013, which is incorporated herein by reference.

### FIELD OF THE INVENTION

The present invention relates to an apparatus for measuring a three-dimensional (3D) shape of an underwater object, and more particularly, to an apparatus and method for measuring a shape of an underwater object, which irradiate a laser point (or laser line) on an underwater target object, capture the underwater object with a charge-coupled device (CCD) camera disposed outside a water tank, recognize the laser point irradiated on the actual target object from an image captured by the CCD camera, and acquire actual absolute coordinates information from a relational formula between actual absolute coordinates and pixel coordinates of the laser point to restore a 3D shape of the target object to be measured in shape, thereby accurately measuring 3D shapes of breasts or the like of an underwater user to be examined.

### BACKGROUND OF THE INVENTION

At present, various methods are used for measuring a 3D shape of an object. For example, the methods include: a method that emits a laser (or an ultrasonic wave, a microwave, or the like), measures a time for which the laser is reflected back from a target object, calculates a distance with the measured time, and restores a shape of the target object by using the calculated distance; a method that irradiates certain light (for example, pattern light, slit light, point light, or the like) on a target object, captures the target object with a camera, and restores a shape of the target object in a light trigonometry or the like; and a method that directly obtains shape information on a target object with a stereo camera.

However, when a target object to be measured is under water, it is difficult to apply the methods. This is because, when a target object is under water, many problems, such as refraction of light passing through dual mediums, reflection of light by an outer wall of a water tank, generation of a false image by a mirror effect of a water surface in the water tank, etc., are caused.

In a breast cancer image diagnosis apparatus using microwaves, a computed tomography (CT) scan is carried out when breasts of a user to be examined are under certain liquid. In this case, shape information on the breasts of the user to be examined is very useful for restoring a high-precision breast tomographic image at a high speed. However, since the breasts of the user to be examined are under the certain liquid, there are limitations in accurately measuring 3D shapes of the breasts through the existing method.

### SUMMARY OF THE INVENTION

In view of the above, the present invention provides an apparatus and method for measuring a shape of an underwater object, which irradiate a laser point (or laser line) on an underwater target object, capture the underwater object with a charge-coupled device (CCD) camera disposed outside a water tank, recognize the laser point irradiated on the actual target object from an image captured by the CCD camera, and acquire actual absolute coordinates information from a relational formula between actual absolute coordinates and pixel coordinates of the laser point to restore a 3D shape of the target object to be measured in shape, thereby accurately measuring 3D shapes of breasts or the like of an underwater user to be examined.

In accordance with a first aspect of the present invention, there is provided an apparatus for measuring a shape of an underwater object, including: a laser emitter configured to irradiate a laser on a surface of a measurement target object under water; a camera configured to capture a laser point generated at the surface of the measurement target object by the laser emitter; and a shape restorer configured to convert a pixel coordinates value of the laser point captured by the camera into an absolute coordinates value to restore a three-dimensional (3D) shape of the measurement target object.

Further, the laser emitter may be disposed in plurality around the measurement target object.

Further, the camera may be disposed in plurality around the measurement target object in correspondence with the laser emitter.

Further, the shape restorer may acquire a plurality of shape information on the measurement target object through the plurality of laser emitters and cameras disposed around the measurement target object.

Further, the shape restorer may interpolate the absolute coordinates value to restore the 3D shape of the measurement target object.

Further, the shape restorer may convert the pixel coordinates value of the laser point into the absolute coordinates value by using a pre-stored conversion relation formula.

Further, the apparatus may further comprise a movement unit configured to move the laser emitter and the camera to near the measurement target object.

Further, the measurement target object may be a breast of a user to be examined, the breast being under water in a water tank.

In accordance with a second aspect of the present invention, there is provided a method of measuring a shape of an underwater object, including: irradiating, by a laser emitter, a laser on a surface of a measurement target object under water; capturing, by a camera, a laser point generated at the surface of the measurement target object by the irradiated laser; extracting a pixel coordinates value of the laser point from the captured image; converting the pixel coordinates value of the laser point into an absolute coordinates value; and restoring a three-dimensional (3D) shape of the measurement target object by using the converted absolute coordinates value.

Further, the laser emitter may be disposed in plurality around the measurement target object.

Further, the camera may be disposed in plurality around the measurement target object in correspondence with the laser emitter.

Further, the restoring may comprise acquiring a plurality of shape information on the measurement target object through the plurality of laser emitters and cameras disposed around the measurement target object.

Further, the restoring may comprise interpolating the absolute coordinates value to restore the 3D shape of the measurement target object.

Further, the converting of the pixel coordinates value may comprise converting the pixel coordinates value of the laser point into the absolute coordinates value according to a pre-stored conversion relation formula.

In accordance with an embodiment of the present invention, a laser point (or laser line) on an underwater target object is irradiated, the underwater object with a charge-coupled device (CCD) camera disposed outside a water tank is captured, the laser point irradiated on the actual target object from an image captured by the CCD camera is recognized, and actual absolute coordinates information from a relational formula between actual absolute coordinates and pixel coordinates of the laser point is acquired to restore a 3D shape of the target object to be measured in shape, thereby accurately measuring 3D shapes of breasts or the like of an underwater user to be examined.

### BRIEF DESCRIPTION OF THE DRAWINGS

The above and other objects and features of the present invention will become apparent from the following description of embodiments given in conjunction with the accompanying drawings, in which:

FIG. 1 a block diagram illustrating an apparatus for measuring a shape of an underwater object in accordance with an embodiment of the present invention;

FIG. 2 is a refraction compensation relation diagram applied to measurement of a shape of an underwater object in accordance with an embodiment of the present invention;

FIG. 3 is a flow chart illustrating a method of measuring a shape of an underwater object in accordance with an embodiment of the present invention;

FIGS. 4A and 4B respectively show a front view and a plane view illustrating a microwave breast cancer tomograph using the method of measuring a shape of an underwater object in accordance with an embodiment of the present invention;

FIG. 5 is an exemplary diagram showing a graph in which pixel coordinates of a laser point are converted into actual absolute coordinates in accordance with an embodiment of the present invention; and

FIG. 6 is a conversion relation diagram between a pixel coordinates value (u) of a laser point and an actual absolute coordinates (x, z) value in accordance with an embodiment of the present invention.

### DETAILED DESCRIPTION OF THE EMBODIMENTS

Hereinafter, the operational principle of the present invention will be described in detail with reference to the accompanying drawings. In the following description of the present invention, if the detailed description of the already known structure and operation may confuse the subject matter of the present invention, the detailed description thereof will be omitted. The following terms are terminologies defined by considering functions in the embodiments of the present invention and may be changed operators intend for the invention and practice. Hence, the terms need to be defined throughout the description of the present invention.

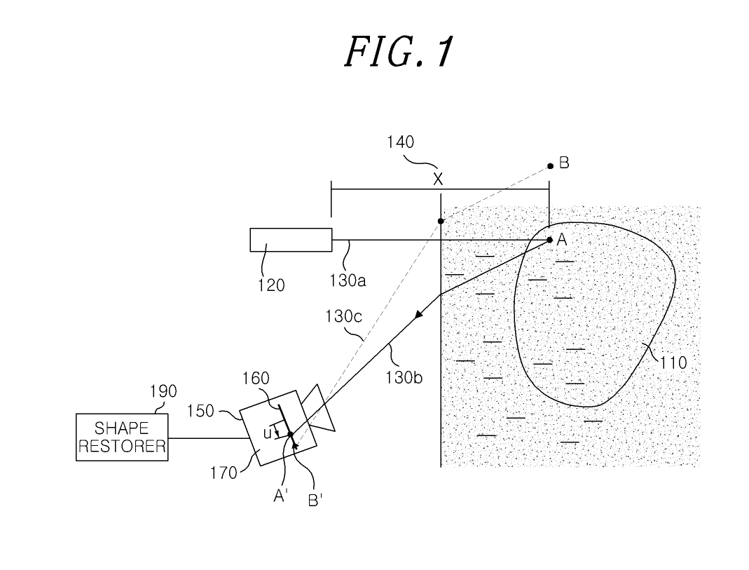

FIG. 1 a block diagram illustrating a configuration of an apparatus for measuring a shape of an underwater object in accordance with an embodiment of the present invention. The apparatus for measuring a shape of an underwater object in accordance with an embodiment of the present invention may include a laser emitter 120, a camera 150, and a shape restorer 190.

Hereinafter, an operation of each element of the apparatus for measuring a shape of an underwater object in accordance with an embodiment of the present invention will be described in detail with reference to FIG. 1.

First, the laser emitter 120 emits a laser on a surface of a measurement target object 110 under water to generate a contactless mark A such as a laser point. At this time, for example, the measurement target object 110 may be a breast of a user to be examined for breast cancer.

The camera 150 captures the mark A which has been generated on the measurement target object 110 by the laser emitter 120.

Each of the laser emitter 120 and camera 150 may be disposed in plurality around the measurement target object 110, for measuring a 3D shape. The apparatus of the present invention may further include a movement unit (not shown) that moves the laser emitter 120 and the camera 150 to near the measurement target object 110 so as to enable measurement of the measurement target object 110.

The shape restorer 190 receives a digital image captured by the camera 150 to process and calculate data. Specifically, the shape restorer 190 recognizes a point A′, at which the mark A on the surface of the measurement target object 110 is disposed, from the digital image captured by the camera 150, and ignores a false image B′ generated by reflection due to a water surface. Also, the shape restorer 190 compensates for refraction of a laser passing through dual mediums.

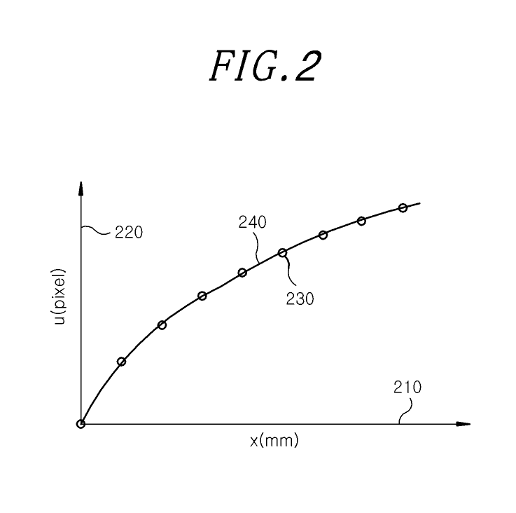

FIG. 2 is a refraction compensation relation diagram applied to measurement of a shape of an underwater object in accordance with an embodiment of the present invention.

Hereinafter, referring to FIG. 2, an absolute distance from the laser emitter 120 to the mark A disposed on the surface of the measurement target object 110 is assumed to be “x” (value to calculate), and the number of pixels between a pixel reference point of a camera CCD 160 and the point A′ with the mark A disposed thereat is assumed to be “u” (measured value). In this case, refraction distortion 130b occurs because the laser passes through the dual mediums, and thus, there is a nonlinear characteristic between the values “x” and “u”.

For this reason, since it is impossible to perform calculation based on the existing light trigonometry, the present invention experimentally measures an actual absolute distance 210 and a pixel distance 220 in advance. Furthermore, the present invention induces a certain function 240 passing through the measured data 230, and applies the function 240 as a conversion formula for converting pixel coordinates of the mart A and actual absolute coordinates to correct refraction distortion.

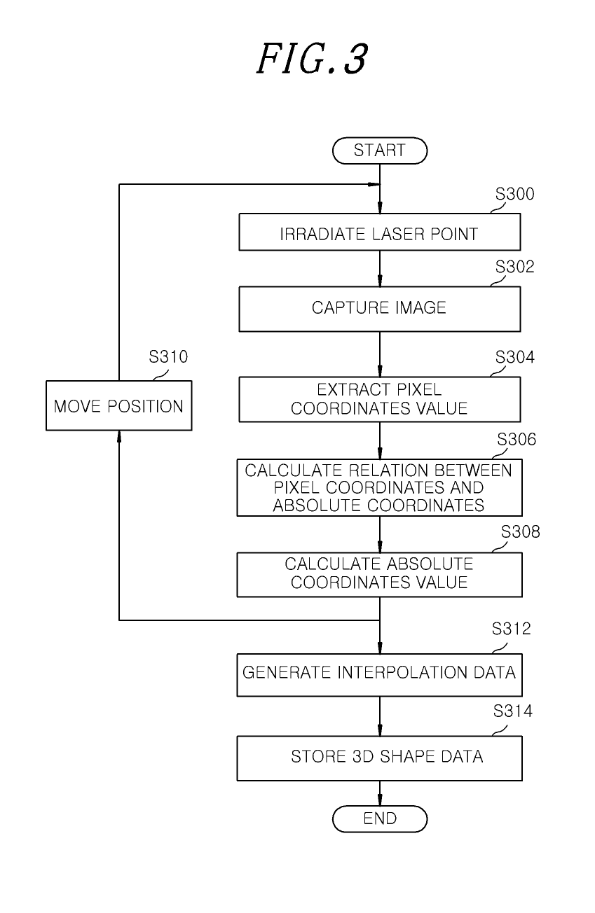

FIG. 3 is an operational control flow chart illustrating a method of measuring a shape of an underwater object in accordance with an embodiment of the present invention. Hereinafter, an embodiment of the present invention will be described in detail with reference to FIGS. 1 to 3.

First, when measurement of an underwater object is started, for example, the laser emitter 120 emits a laser on the measurement target object 110 under water with which a water tank is filled, and thus generates the mark A such as a laser point in operation S300.

Like this, when the mark A is generated on the surface of the measurement target object 110 by the laser emitter 120, the camera 150 captures the mark A generated on the surface of the measurement target object 110 to capture an image including the mark A in operation S302.

Like this, when the image including the mark A generated on the surface of the measurement target object 110 is captured by the camera 150, the shape restorer 190 recognizes the point A′ corresponding to the mark A to extract a pixel coordinates value of the point A′ in operation S304.

Subsequently, the shape restorer 190 calculates a relation between a pre-stored pixel coordinates value and an actual absolute coordinates value in operation S306, and calculates an absolute coordinates value of the point A′ corresponding to the extracted pixel coordinates value in operation S308.

Like this, when the absolute coordinates value of the point A′ is calculated, the laser emitter 120 and the camera 150 are moved in positions in operation S310, and operations S300 to S308 are repeated, thereby calculating a plurality of shape information data.

In operation S312, the shape restorer 190 interpolates the plurality of shape information data obtained through operations S300 to S308.

Subsequently, the shape restorer 190 stores 3D shape data information (generated through the data interpolation) on the measurement target object in operation S314.

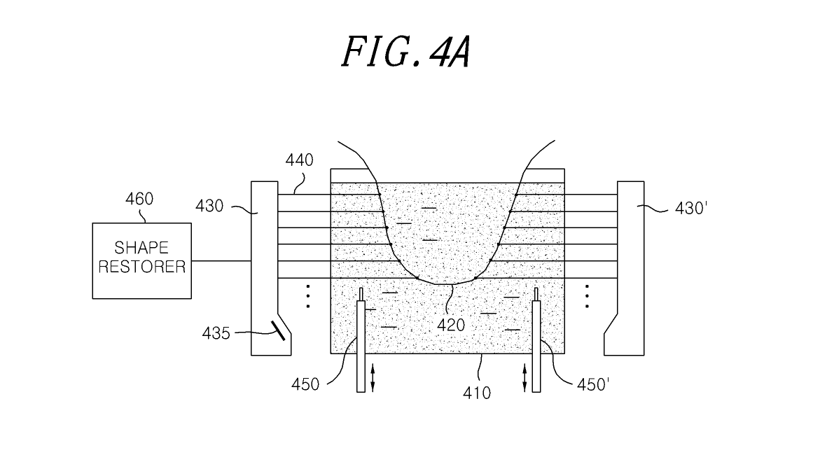

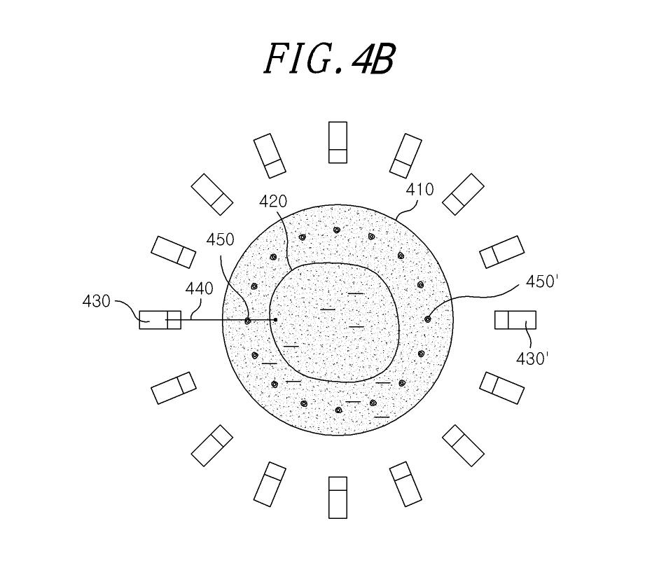

FIGS. 4A and 4B respectively show a front view and a plane view illustrating a microwave breast cancer tomograph using the method of measuring a shape of an underwater object in accordance with an embodiment of the present invention.

Referring to FIGS. 4A and 4B, the microwave breast cancer tomograph may include a water tank 410 containing certain liquid and a plurality of transmitting/receiving antennas 450 and 450′ disposed around a breast of a user to be examined. According to the present invention, a plurality of vertically disposed laser emitters and one camera 435 are integrated into each of shape measurement modules 430 and 430′, and for example, sixteen shape measurement modules 430 and 430′ may be disposed around the water tank 410.

A measuring sequence is based on the above description. To provide an additional description, the shape measurement modules 430 and 430′ sequentially irradiate a laser 440 on a breast 420 of the user to be examined, and the camera 435 captures images.

Subsequently, a shape restorer 460 connected to the shape measurement modules 430 and 430′ detects a pixel of a mark, generated by the laser emitter of each of the shape measurement modules 430 and 430′, from each of the images captured by the camera 435, and converts coordinates of the pixel into actual absolute coordinates by using a predetermined conversion relation diagram. The shape restorer 460 interpolates the finally converted actual absolute coordinates to restore an entire 3D breast shape.

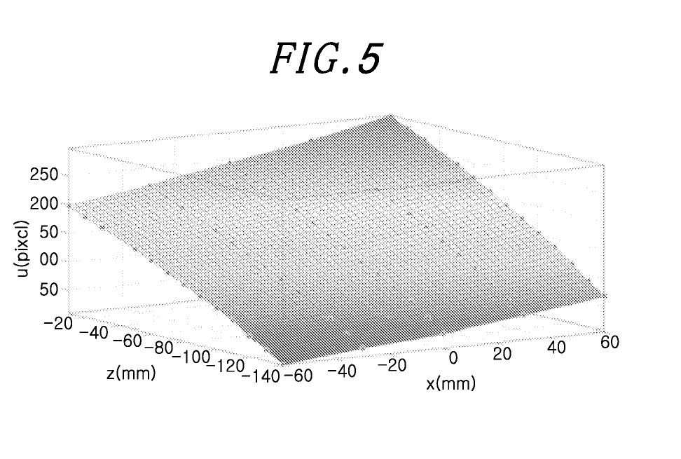

FIG. 5 shows a graph in which pixel coordinates of a laser mark measured by the shape measurement module are converted into actual absolute coordinates in accordance with an embodiment of the present invention. A round marker in the graph of FIG. 5 is pixel data of a mark (measured by the camera 435) generated by a laser.

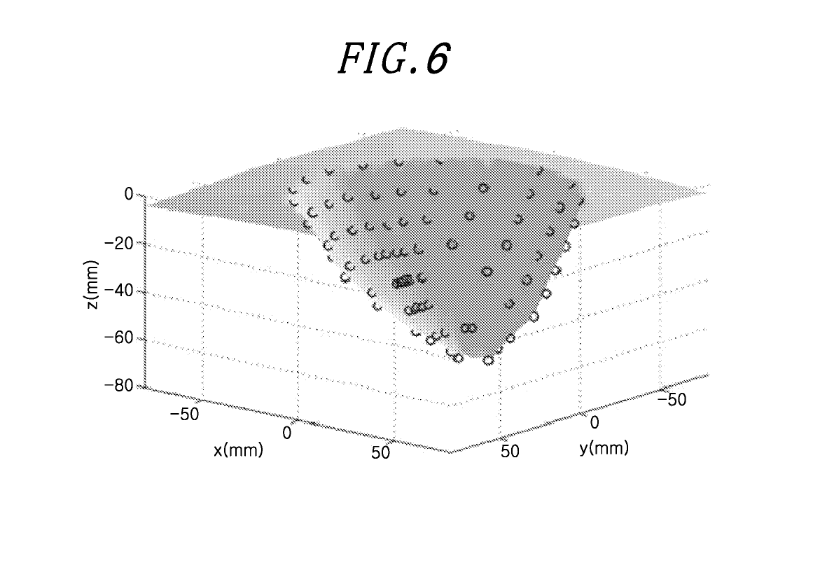

FIG. 6 shows a conversion relation diagram between a pixel coordinates value (u) of a laser point and an actual absolute coordinates (x, z) value in accordance with an embodiment of the present invention.

The present invention may convert pixel coordinates of a laser mark into actual absolute coordinates by using the conversion relation diagram of FIG. 6, and then, by performing data interpolation, restore an entire 3D breast shape.

As described above, in the apparatus for measuring a 3D shape of an underwater object, the present invention irradiates a laser point (or laser line) on an underwater target object, captures the underwater object with a charge-coupled device (CCD) camera disposed outside a water tank, recognizes the laser point irradiated on the actual target object from an image captured by the CCD camera, and acquires actual absolute coordinates information from a relational formula between actual absolute coordinates and pixel coordinates of the laser point to restore a 3D shape of the target object to be measured in shape, thereby accurately measuring 3D shapes of breasts or the like of an underwater user to be examined.

While the invention has been shown and described with respect to the embodiments, the present invention is not limited thereto. It will be understood by those skilled in the art that various changes and modifications may be made without departing from the scope of the invention as defined in the following claims.

What is claimed is:

1. An apparatus for measuring a shape of an underwater object, comprising:
a laser emitter configured to irradiate a laser on a surface of a measurement target object under water;
a camera configured to capture a laser point generated at the surface of the measurement target object by the laser emitter; and
a shape restorer configured to convert a pixel coordinates value of the laser point captured by the camera into an absolute coordinates value to restore a three-dimensional (3D) shape of the measurement target object.

2. The apparatus of claim 1, wherein the laser emitter is disposed in plurality around the measurement target object.

3. The apparatus of claim 2, wherein the camera is disposed in plurality around the measurement target object in correspondence with the laser emitter.

4. The apparatus of claim 1, wherein the shape restorer acquires a plurality of shape information on the measurement target object through the plurality of laser emitters and cameras disposed around the measurement target object.

5. The apparatus of claim 1, wherein the shape restorer interpolates the absolute coordinates value to restore the 3D shape of the measurement target object.

6. The apparatus of claim 1, wherein the shape restorer converts the pixel coordinates value of the laser point into the absolute coordinates value by using a pre-stored conversion relation formula.

7. The apparatus of claim 1, further comprising a movement unit configured to move the laser emitter and the camera to near the measurement target object.

8. The apparatus of claim 1, wherein the measurement target object is a breast of a user to be examined, the breast being under water in a water tank.

9. A method of measuring a shape of an underwater object, comprising:
irradiating, by a laser emitter, a laser on a surface of a measurement target object under water;
capturing, by a camera, a laser point generated at the surface of the measurement target object by the irradiated laser;
extracting a pixel coordinates value of the laser point from the captured image;
converting the pixel coordinates value of the laser point into an absolute coordinates value; and
restoring a three-dimensional (3D) shape of the measurement target object by using the converted absolute coordinates value.

10. The method of claim 9, wherein the laser emitter is disposed in plurality around the measurement target object.

11. The method of claim 10, wherein the camera is disposed in plurality around the measurement target object in correspondence with the laser emitter.

12. The method of claim 9, wherein the restoring comprises acquiring a plurality of shape information on the measurement target object through the plurality of laser emitters and cameras disposed around the measurement target object.

13. The method of claim 9, wherein the restoring comprises interpolating the absolute coordinates value to restore the 3D shape of the measurement target object.

14. The method of claim 9, wherein the converting of the pixel coordinates value comprises converting the pixel coordinates value of the laser point into the absolute coordinates value according to a pre-stored conversion relation formula.
